(19) United States
(12) Patent Application Publication
(10) Pub. No.: US 2012/0172818 A1
(43) Pub. Date: Jul. 5, 2012

(54) Medical Device Having a Mechanism and Use of a Low-Friction Synthetic Material Within a Medical Device

(75) Inventors: Michael Harms, Frankfurt am Main (DE); Steffen Raab, Frankfurt am Main (DE); Uwe Dasbach, Frankfurt am Main (DE)

(73) Assignee: SANOFI-AVENTIS DEUTSCHLAND GMBH, Frankfurt am Main (DE)

(21) Appl. No.: 13/254,087

(22) PCT Filed: Mar. 31, 2010
(86) PCT No.: PCT/EP10/54349

§ 371 Date: Mar. 16, 2012

Related U.S. Application Data
(60) Provisional application No. 61/169,872, filed on Apr. 16, 2009.

(30) Foreign Application Priority Data
Mar. 31, 2009 (EP) 09004672.3

(51) Int. Cl. A61M 5/315; A61M 5/31

(52) U.S. Cl. 604/230; 604/187

(57) ABSTRACT

A first movable element (4) and a second movable element (7) of a mechanism in a medical device are arranged in such a manner that, during an operation of the mechanism, a surface of the first element slides on a surface of the second element. The first element and the second element are formed from materials providing a coefficient of sliding friction of said surfaces on one another of less than 0.14 at a relative velocity of 2 mm per second.

This invention relates to the use of synthetic or plastic material of low friction in a mechanism of a medical device, especially a drug delivery device or injection device.

The application of mechanisms in medical devices is accompanied with an increasing demand for mechanical components or elements of specialized shapes that can be manufactured in large quantities. Synthetic or plastic materials are ideally suited for this purpose. Portable medical devices that are used by patients and have to be everywhere available are provided with mechanisms of reduced dimensions. It is important that the mechanism be reliable and the movable elements sufficiently smooth-running. For reasons of easy use, a lubrication is to be avoided.

Portable drug delivery devices are generally known for the administration of a medicinal fluid or drug that is suitable for the self-administration by a patient. A drug injection device is especially useful in the shape of a pen, which can be handled easily and kept everywhere available. A sophisticated type of drug delivery device is constructed to be refillable and reusable many times. A dose of a drug is delivered by means of a drive mechanism, which also allows to set the amount of fluid to be thus injected.

EP 1 923 083 A1 describes a drug delivery device in the shape of an injection pen having a drive mechanism, which allows to deliver a plurality of different prescribed doses.

It is an object of the present invention to disclose a means of providing an improved mechanism of a medical device with movable elements.

This object is achieved with the medical device according to claim 1 and with the use of a low-friction synthetic material according to claim 12, respectively. Further aspects and variations of the invention derive from the depending claims.

The medical device comprises a mechanism with movable elements provided for operating the device. A first movable element and a second movable element are arranged in such a manner that, during an operation of the mechanism, a surface of the first element slides on a surface of the second element. The first element and the second element are formed from materials providing a coefficient of sliding friction of said surfaces on one another of less than 0.14 at a relative velocity of 2 mm per second.

The friction between rough planar surfaces of two bodies that are in contact and move relatively to one another, so that the surfaces slide on one another, generates a force F_{R} of a retarding effect directed within the plane of the surfaces, thus decreasing the velocity of the relative movement. At a certain specified relative speed of the bodies, the absolute value of the frictional force F_{R} can generally be regarded as being proportional to the absolute value of a force F_{N} perpendicular to the plane of the surfaces, by which the bodies are pressed on one another. The quotient of the absolute value of the frictional force F_{R} and the absolute value of the perpendicular force F_{N} is called coefficient p of sliding friction, so that the equality F_{R}=μ(vᵣ)×F_{N} is supposed for any specified relative velocity yᵣ of the bodies.

In an embodiment of the medical device, the coefficient of sliding friction of the sliding surfaces of the first element and of the second element is less than 0.10 at a relative velocity of 2 mm per second.

In a further embodiment of the medical device, the coefficient of sliding friction of the sliding surfaces of the first element and of the second element is less than 0.08 at a relative velocity of 2 mm per second.

In a further embodiment of the medical device, the material of at least one of the first element and the second element is a polybutylene terephthalate.

In a further embodiment of the medical device, the material of at least one of the first element and the second element is a polyoxymethylene.

In a further embodiment of the medical device, the material of at least one of the first element and the second element is a liquid crystalline polymer.

In a further embodiment of the medical device, the first element is a drive sleeve and the second element is a nut.

In a further embodiment of the medical device, the first element is a drive sleeve and the second element is a piston rod.

In a further embodiment of the medical device, the first element is a nut and the second element is a piston rod.

In a further embodiment of the medical device, the first element is an operation button and the second element is a washer.

The medical device can particularly be a drug delivery device or an injection device, especially a portable injection device having the shape of a pen. Since such an injection device or injection pen is designed to be handy and everywhere available, the mechanism provided for the operation of the injection device has to be arranged within restricted dimensions. It is therefore advantageous to equip the mechanism with small sliding elements of low-friction materials.

The invention further discloses the use of at least one low-friction synthetic material within a mechanism of a medical device. The low-friction synthetic material is used in conjunction with the same or with a further material providing a coefficient of sliding friction of less than 0.14 at a relative velocity of 2 mm per second.

The low-friction synthetic material can especially be a polybutylene terephthalate, a polyoxymethylene, or a liquid crystalline polymer.

Owing to their surface properties, the low-friction synthetic materials are suitable for a manufacturing of mechanical elements having surfaces that are smooth-running in a sliding contact with a surface of the same or a suitably selected further material and render the desired low friction.

Further aspects and examples of the invention are described in conjunction with the appended figures.

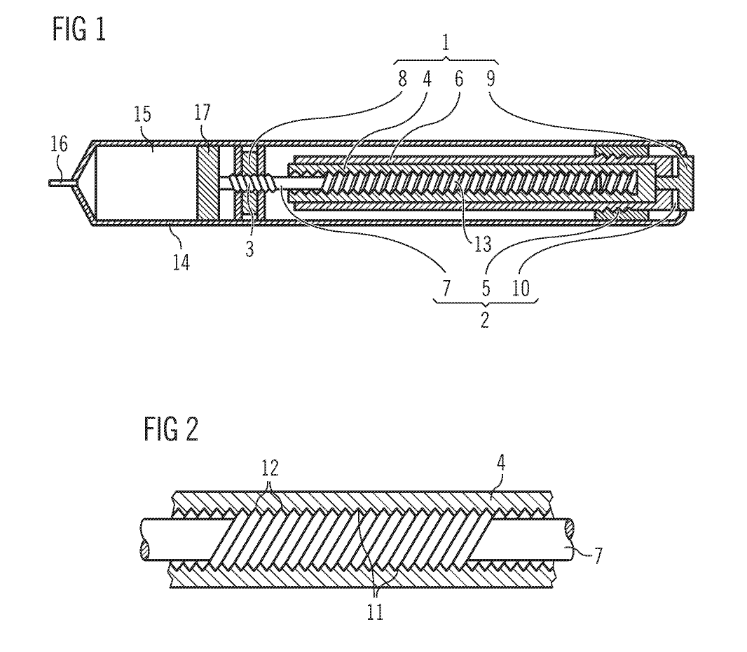

FIG. 1 shows a cross-section of an injection pen having a mechanism.

FIG. 2 shows a cross-section of sliding elements of the mechanism.

FIG. 1 shows a cross-section of an injection device in the shape of a pen with a mechanism inside a housing or body 14. A proximal end is provided with an operation button 9, and a distal end is provided with a reservoir 15 provided for a drug or pharmaceutical fluid that is to be injected through a needle 16. The delivery of the drug is effected by means of a piston 17, which is moved by a piston rod 7 in the direction of the longitudinal extension of the device, thus reducing the volume of the reservoir 15 according to the doses to be administered. The reservoir 15 can be provided for the insertion of a cartridge containing the drug. In this case, the piston 17 is moved in the cartridge and the piston rod 7 moves through a hole in the bottom of the cartridge.

The term “drug or pharmaceutical fluid”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl— ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ωcarboxyheptadecanoyl)-des(B30) human insulin and B29-N-(w-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAs28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group —Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38[Asp28] Exendin-4(1-39)-NH2,

H-Asn-(Glu)5des Pro36, Pro37, Pro38 [Asp28] Exendin-4(1-39)-NH2,

des Pro36, Pro37, Pro38, [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38, [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38, [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38, [Met(O)14, Trp(O2)25] Exendin-4(1-39)-NH2,

H-(Lys)6-des Pro36, Pro37, Pro38, [Met(O)14, Asp28] Exendin-4(1-39)-NH2,

H-Asn-(Glu)5-des Pro36, Pro37, Pro38, [Met(O)14, Trp(O2)25, Asp28] Exendin-4(1-39)-NH2,

des Pro36, Pro37, Pro38, [Met(O)14, Trp(O2)25, Asp28] Exendin-4(1-39)-(Lys)6-NH2,

H-(Lys)6-des Pro36, Pro37, Pro38, [Met(O)14, Trp(O2)25, Asp28] Exendin-4(S1-39)-(Lys)6-NH2,

H-Asn-(Glu)5-des Pro36, Pro37, Pro38, [Met(O)14, Trp(O2)25, Asp28] Exendin-4(1-39)-(Lys)6-NH2;

or a pharmaceutically acceptable salt or solvate of any one of the afore-mentioned Exendin-4derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The embodiment according to FIG. 1 shows several examples of pairs of movable elements 1, 2 which have surfaces sliding on one another when the mechanism is operated. The piston rod 7 carries a screw thread 3 and is surrounded by a piston rod nut 8, which has a thread of the same pitch on the inner wall of a hole through its centre. The piston rod 7 and the piston rod nut 8 are interlocked by the screw thread 3 and can be rotated relatively to one another. Simultaneously with the rotation, the screw thread 3 generates an axial relative movement resulting in an overall helical relative movement. The piston rod 7 and the piston rod nut 8 thus form a pair of sliding elements. The friction between these elements is reduced if they are formed from low-friction synthetic materials. The piston rod 7 can be a liquid crystalline polymer, for example, and the piston rod nut 8 a polyoxymethylene, for example.

The piston rod 7 can be driven by means of a drive sleeve 4, which has a thread fitting into a further screw thread 13 of the piston rod 7. The drive sleeve 4 and the piston rod 7 form another pair of sliding elements with the surfaces of the threads sliding on one another.

FIG. 2 shows an enlarged cross-section of the piston rod 7 and the drive sleeve 4. The further screw thread 13 of the piston rod 7 and the corresponding thread of the drive sleeve 4 form a first surface 11 and a second surface 12 sliding on one another when the drive sleeve 4 is helically rotated relatively to the piston rod 7. In order to reduce the friction between these elements, they can also be formed from low-friction synthetic materials. If the piston rod 7 is a liquid crystalline polymer, as in the aforementioned example, the drive sleeve 4 can be a further polyoxymethylene, for example.

The dosage is effected by a part of the mechanism that comprises a further drive sleeve 6 and a dial nut 5 surrounding the further drive sleeve 6. The further drive sleeve 6 has a screw thread and the dial nut 5 has an inner thread of the same pitch. The further drive sleeve 6 and the dial nut 5 are interlocked by the threads and can be rotated relatively to one another in a helical movement, so that they also form a pair of sliding elements. The friction between these elements can also be reduced by the use of low-friction synthetic materials. The further drive sleeve 6 can be a polybutylene terephthalate, for example, and the dial nut 5 a polyoxymethylene, for example.

The mechanism, especially the further drive sleeve 6, is operated by an operation button 9. The operation button 9 slides on a washer 10 when the further drive sleeve 6 or some intermediate element is rotated relatively to the operation button 9, which can be kept rotationally fixed with respect to the body 14. In order to reduce the friction between these elements, they can also be formed using a low-friction synthetic material. It is preferred to have a metallic operation button 9. If the operation button 9 is aluminum, for example, the washer 10 can be a polyoxymethylene, for example. The coefficient of sliding friction of aluminum and polyoxymethylene on one another is less than 0.14 at a relative velocity of 2 mm per second.

Commercially available low-friction synthetic materials that can be favorably used in a medical device like the one shown in FIG. 1 are, for instance, the following:

polybutylene terephthalate:

Celanex®2404MT or Celanex®2404MT 20/9107 white, manufactured by Ticona; polyoxymethylene:
a) POM: MT8F02 (used for dial nut 5 and washer 10, for example): Hostaform® MT8F02 natural-coloured, manufactured by Ticona,
b) POM: MTF01 (used for piston rod nut 8, for example): Hostaform® MT8F01 natural-coloured, manufactured by Ticona, and
c) POM: MT12U01 (used for drive sleeve 4, for example): Hostaform®MT12U01 natural-coloured, manufactured by Ticona; liquid crystalline polymer:

LCP: MT1335 (used for piston rod 7, for example):

Vectra®MT1335 natural-coloured, combined with

Masterbatch: LKX1057 black, both manufactured by Ticona.

### REFERENCE NUMERALS

1 first element

2 second element

3 screw thread

4 drive sleeve

5 dial nut

6 further drive sleeve

7 piston rod

8 piston rod nut

9 operation button

10 washer

11 surface of the second element

12 surface of the first element

13 screw thread

14 body

15 reservoir

16 needle

17 piston

1. A medical device comprising:
a mechanism provided for operating the device, the mechanism comprising a first movable element and a second movable element;
the first movable element and the second movable element being arranged in such a manner that, during an operation of the mechanism, a surface of the first element slides on a surface of the second element; and
the first element and the second element being formed from materials providing a coefficient of sliding friction of said surfaces on one another of less than 0.14 at a relative velocity of 2 mm per second.

2. The medical device according to claim 1, wherein the first element and the second element are formed from materials providing a coefficient of sliding friction of said surfaces on one another of less than 0.10 at a relative velocity of 2 mm per second.

3. The medical device according to claim 1, wherein the first element and the second element are formed from materials providing a coefficient of sliding friction of said surfaces on one another of less than 0.08 at a relative velocity of 2 mm per second.

4. The medical device according to claim 1, wherein the material of at least one of the first element and the second element is a polybutylene terephthalate.

5. The medical device according to claim 1, wherein the material of at least one of the first element and the second element is a polyoxymethylene.

6. The medical device according to claim 1, wherein the material of at least one of the first element and the second element is a liquid crystalline polymer.

7. The medical device according to claim 1, wherein the first element is a drive sleeve and the second element is a nut.

8. The medical device according to claim 1, wherein the first element is a drive sleeve and the second element is a piston rod.

9. The medical device according to claim 1, wherein the first element is a nut and the second element is a piston rod.

10. The medical device according to claim 1, wherein the first element is an operation button and the second element is a washer.

11. The medical device according to claim 1, wherein the device is a pen-type drug delivery device or injection device.

12. Use of a low-friction synthetic material within a mechanism of a medical device in conjunction with the same or with a further material providing a coefficient of sliding friction of less than 0.14 at a relative velocity of 2 mm per second.

13. The use of a low-friction synthetic material according to claim 12, wherein the low-friction synthetic material is a polybutylene terephthalate.

14. The use of a low-friction synthetic material according to claim 12, wherein the low-friction synthetic material is a polyoxymethylene.

15. The use of a low-friction synthetic material according to claim 12, wherein the low-friction synthetic material is a liquid crystalline polymer.
